(19) United States
(12) Patent Application Publication
(10) Pub. No.: US 2021/0162174 A1
(43) Pub. Date: Jun. 3, 2021

(54) METHOD AND SYSTEM FOR TRANSITIONING BETWEEN STATES IN FLEXIBLE ROBOT DEVICES

(71) Applicant: Intuitive Surgical Operations, Inc., Sunnyvale, CA (US)

(72) Inventor: Nicola Diolaiti, Menlo Park, CA (US)

(73) Assignee: Intuitive Surgical Operations, Inc., Sunnyvale, CA (US)

(21) Appl. No.: 17/104,679

(22) Filed: Nov. 25, 2020

Related U.S. Application Data
(60) Provisional application No. 62/941,996, filed on Nov. 29, 2019.

(51) Int. Cl. A61M 25/01; A61B 34/35

(52) U.S. Cl. CPC A61M 25/0113; A61B 2034/301; A61B 34/35

(57) ABSTRACT

A medical system involves a flexible catheter, an instrument carriage, an actuator, and a controller coupled to the actuator. The flexible catheter includes a flexible body having a proximal portion and a distal portion. The distal portion is articulable along an articulation degree of freedom of the flexible catheter. The instrument carriage supports the flexible catheter at the proximal portion of the flexible body. The actuator is disposed on the instrument carriage and configured to drive the distal portion of the flexible catheter along the articulation degree of freedom. The controller is configured to detect an exception of the medical system, based on detection of the exception, switch from an operating state to a control state, and control the actuator, in the control state, to relax the flexible catheter along the articulation degree of freedom.

### CROSS-REFERENCE TO RELATED APPLICATIONS

This application claims priority to U.S. Provisional Patent Application No. 62/941,996 filed Nov. 29, 2019, the entire disclosure of which is hereby expressly incorporated by reference herein.

### BACKGROUND

### Field of Invention

The present invention generally provides improved robotic and/or medical (including surgical) devices, systems, and methods.

### Overview

Flexible devices may be used to perform tasks at worksites, such as when the worksites are accessible through one or more orifices (e.g., one or more openings in a human, animal, machinery, etc.). For example, a minimally invasive medical procedure may be performed through an orifice to reduce an amount of tissue that is damaged during the medical procedure, thereby reducing patient recovery time, discomfort, and harmful side effects. The orifice may be a natural orifice in a patient anatomy or a surgical incision. An operator (e.g., a physician, a physician assistant, a surgeon, etc.) may insert a minimally invasive medical instrument (including surgical, diagnostic, therapeutic, and/or biopsy instruments) through the orifice to reach a target tissue location. A flexible and/or steerable elongate device, such as a flexible catheter, may be used to reach a region of interest within the patient anatomy via an anatomic passageway. Control of such an elongate device by an operator may involve the management of multiple degrees of freedom including insertion and retraction of the elongate device with respect to the patient anatomy and steering of the device. Versatile systems and methods for instrument control are needed to address these and other challenges.

### SUMMARY

In general, in one aspect, one or more embodiments relate to a medical system, comprising: a flexible catheter, comprising: a flexible body having a proximal portion and a distal portion, wherein the distal portion is articulable along an articulation degree of freedom of the flexible catheter; an instrument carriage supporting the flexible catheter at the proximal portion of the flexible body; a first actuator disposed on the instrument carriage and configured to drive the distal portion of the flexible catheter along the articulation degree of freedom; and a controller coupled to the first actuator, the controller configured to: detect an exception of the medical system; based on detection of the exception, switch from an operating state to a control state; and control the first actuator, in the control state, to relax the flexible catheter along the articulation degree of freedom.

In general, in one aspect, one or more embodiments relate to a method for operating a medical system, the method comprising: when detecting an exception of the medical system: switching from an operating state to a control state; and when in the control state: controlling a first actuator for driving a distal portion of a flexible catheter along an articulation degree of freedom to relax the flexible catheter of the medical system along the articulation degree of freedom.

In general, in one aspect, one or more embodiments relate to a non-transitory machine-readable medium comprising a plurality of machine-readable instructions executed by one or more processors associated with a coordination system, the plurality of machine-readable instructions causing the one or more processors to perform a method comprising: when detecting an exception of the medical system: switching from an operating state to a control state; and when in the control state: controlling a first actuator for driving a distal portion of a flexible catheter along an articulation degree of freedom to relax the flexible catheter of the medical system along the articulation degree of freedom.

Other aspects of the invention will be apparent from the following description and the appended claims.

### BRIEF DESCRIPTION OF DRAWINGS

FIG. 1A diagrammatically shows a computer-assisted medical system, in accordance with one or more embodiments.

FIG. 1B diagrammatically shows a control system of the computer-assisted medical system, in accordance with one or more embodiments.

FIG. 2 shows an example of a computer-assisted medical system, in accordance with one or more embodiments.

FIG. 3 shows an example of a manipulator assembly including an instrument and a manipulator arm holding the instrument, in accordance with one or more embodiments.

FIG. 4 shows an example of an instrument manipulator including a catheter assembly, in accordance with one or more embodiments.

FIG. 5A and FIG. 5B schematically show examples of computer-assisted medical systems including flexible catheters, in accordance with one or more embodiments.

FIG. 6 shows a medical scenario, in accordance with one or more embodiments.

FIG. 7A, FIG. 7B, and FIG. 7C show an example method for switching between states, in accordance with one or more embodiments.

### DETAILED DESCRIPTION

Specific embodiments of the disclosure will now be described in detail with reference to the accompanying figures. Like elements in the various figures are denoted by like reference numerals for consistency.

In the following detailed description of embodiments of the disclosure, numerous specific details are set forth. However, it will be apparent to one of ordinary skill in the art that some embodiments may be practiced without these specific details. In other instances, well-known features have not been described in detail to avoid unnecessarily complicating the description.

Throughout the application, ordinal numbers (e.g., first, second, third, etc.)

may be used as an adjective for an element (i.e., any noun in the application). The use of ordinal numbers is not to imply or create any particular ordering of the elements nor to limit any element to being only a single element unless expressly disclosed, such as by the use of the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

Although some of the examples described herein refer to medical procedures and medical tools, the examples described herein may apply to medical and non-medical procedures, and to medical and non-medical tools. For example, the tools, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, setting up or taking down the system, and training medical or non-medical personnel. Additional example applications include use for procedures on tissue removed from human or animal anatomies (without return to a human or animal anatomy), and performing procedures on human or animal cadavers. Further, these techniques can also be used for medical treatment or diagnosis procedures that do, or do not, include surgical aspects.

In general, embodiments of the disclosure may enable a transitioning between states in flexible robotic-assisted devices, in accordance with one or more embodiments. A transition from a regular operating state to a different state may occur when an exception is detected. An exception may be, for example, a failure of a system component, inaccurate or noisy sensing signals, unexpected user actions, etc., as discussed below. Depending on the type of exception, different behaviors may occur. For example, detection of an exception may trigger a transition of a flexible robotic-assisted device to a state in which all degrees of freedom are locked to hold the current position or orientation. Alternatively, when a flexible device of a robotic-assisted system is inserted into a human anatomy via an anatomic orifice and the system enters a safe control state, some degrees of freedom may be released or relaxed and/or some degrees of freedom may be locked.

Consider, for example, a bronchoscopy involving a flexible catheter extending into the lungs via the airways. Assume that a failure impairs aspects of the control system for the flexible catheter, such as a sensing signal used for tracking movement of the flexible catheter becoming unavailable. In this scenario, the degrees of freedom of the flexible catheter might not be locked, as this may inhibit removal (or other movement) of the flexible catheter. Instead, the flexible catheter may be relaxed to a limp state. In the limp state, the flexible catheter may passively follow the airways when the flexible catheter is withdrawn, thereby reducing the likeliness of damage to the airways. Flexible robotic-assisted systems, methods for controlling the flexible robotic-assisted systems, and for switching between different operating modes, in accordance with one or more embodiments, are described below.

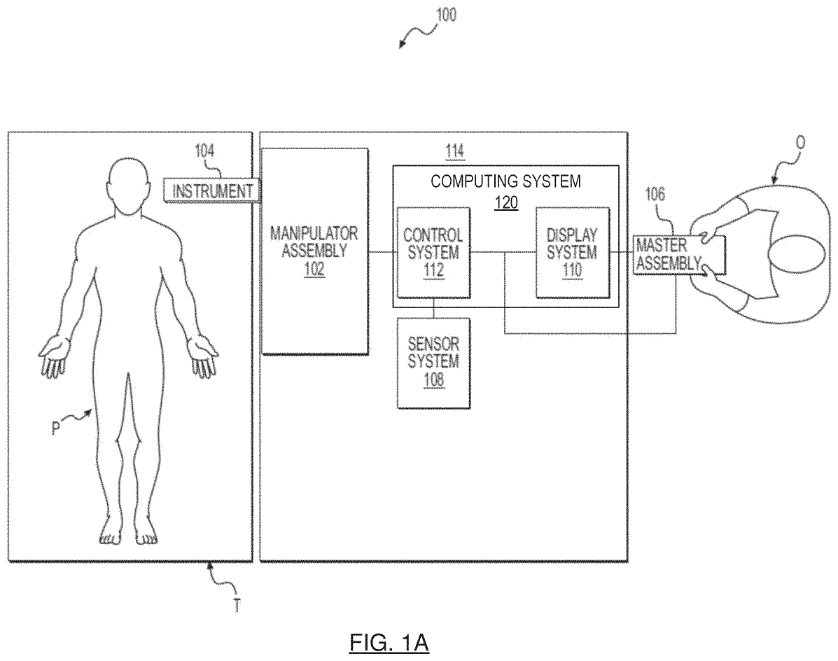

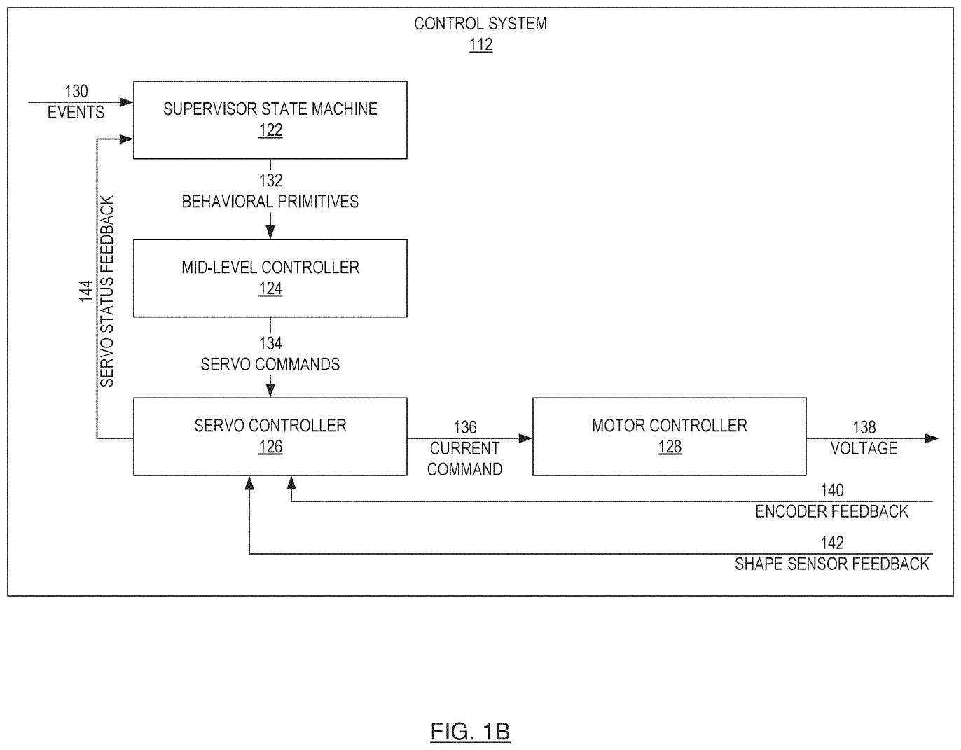

Referring now to the drawings, in which like reference numerals represent like parts throughout the several views, FIG. 1A shows a simplified diagram of a computer-assisted medical system (100) in accordance with one or more embodiments. The computer-assisted medical system (100) may be suitable for use in, for example, surgical, diagnostic, therapeutic, and/or biopsy procedures. While some embodiments are provided herein with respect to such procedures, any reference to medical (e.g., surgical) instruments and medical methods is non-limiting. The systems, instruments, and methods described herein may be used for animals, human cadavers, animal cadavers, portions of human or animal anatomy, non-surgical diagnosis, as well as for industrial systems and general robotic or teleoperational systems.

As shown in FIG. 1A, the computer-assisted medical system (100) generally includes a manipulator assembly (102) for operating a medical instrument (104) to perform various procedures on a patient (P) in accordance with one or more embodiments of the invention. The medical instrument (104) may include a flexible catheter and may be steered when extended into an internal site within the body of the patient (P).

The manipulator assembly (102) and the instrument (104) may be teleoperated, non-teleoperated, or a hybrid teleoperated and non-teleoperated assembly with select degrees of freedom of motion that may be motorized and/or teleoperated and select degrees of freedom of motion that may be non-motorized and/or non-teleoperated. The manipulator assembly (102) may be mounted to an operating table (T), or to a main support (114) (e.g., a cart, stand, second table, and/or the like). A master assembly (106) may allow an operator (e.g., a surgeon, a clinician, or a physician) to view the interventional site and to control the manipulator assembly (102), including the instrument (104).

The master assembly (106) may be located at an operator console which may be located in the same room as operating table (T), such as at the side of a surgical table on which patient (P) is located. However, it should be understood that the operator (O) can be located in a different room or a completely different building from patient (P). The master assembly (106) may include one or more control devices for controlling the manipulator assembly (102). The control devices may include any number of a variety of input devices, such as joysticks, trackballs, data gloves, trigger-guns, hand-operated controllers, voice recognition devices, body motion or presence sensors, and/or the like. To provide the operator (O) a strong sense of directly controlling the instrument (104) the control devices may be provided with the same degrees of freedom as the associated medical instrument (104). In this manner, the control devices provide operator (O) with telepresence or the perception that the control devices are integral with medical instruments (104).

In one or more embodiments, the manipulator assembly (102) supports the medical instrument (104) including the flexible catheter, and may include a kinematic structure of one or more non-servo controlled links (e.g., one or more links that may be manually positioned and locked in place, generally referred to as a set-up structure), and/or one or more servo controlled links (e.g., one or more powered links that may be controlled in response to commands from the control system), and a manipulator. The manipulator assembly (102) may include a plurality of actuators or motors that drive the medical instrument (104) in response to commands from the control system (e.g., a control system (112)).

The actuators may include drive systems that when coupled to the medical instrument (104) may advance the medical instrument (104) into a naturally or surgically created anatomic orifice. Other drive systems may move the distal end of the medical instrument (104) in one or more degrees of freedom, which may include at least a linear insertion/retraction motion and one or more articulation motions to orient the instrument, as discussed in detail below. Additionally, the actuators may be used to actuate an articulable end effector of medical instrument (104) for grasping tissue in the jaws of a biopsy device and/or the like. Actuator sensors (e.g., resolvers, encoders, potentiometers, and other mechanisms) may provide sensor data to the medical system (100) describing the position, rotation, and/or orientation of motor shafts. This actuator sensor data may be used to determine motion of the objects manipulated by the actuators. The actuator sensors may comprise proximal sensors located proximal to other sensors used to measure at a distal portion of the medical instrument or along the medical instrument.

The computer-assisted medical system (100) may include a sensor system (108) with one or more sub-systems for receiving information about the instrument (including the flexible catheter) of manipulator assembly (102). Such sub-systems may include a position/location sensor system (e.g., an electromagnetic (EM) sensor system); a shape sensor system for determining the position, orientation, speed, velocity, pose, and/or shape of a distal portion and/or of one or more segments along a flexible body that may make up the flexible catheter housing the instrument (104); and/or a visualization system for capturing images from the distal end of the instrument (104). One or more sensors of the sensor system (108) may include distal sensors used to measure at a distal portion of the flexible catheter housing the instrument and/or along the flexible catheter.

In one or more embodiments, the computer-assisted medical system (100) also includes a display system (110) for displaying an image or representation of the surgical site and medical instrument (104), including the flexible catheter, generated by sub-systems of sensor system (108), recorded pre-operatively or intra-operatively using image data from imaging technology and/or a real time image such as, computed tomography (CT), magnetic resonance imaging (MRI), fluoroscopy, thermography, ultrasound, optical coherence tomography (OCT), thermal imaging, impedance imaging, laser imaging, nanotube X-ray imaging, endoscopic images, and/or the like. The pre-operative or intra-operative image data may be presented as two-dimensional, three-dimensional, or four-dimensional (including e.g., time based or velocity-based information) images and/or as images from models created from the pre-operative or intraoperative image data sets. The display system (110) and the master assembly (106) may be oriented so the operator (O) can control the medical instrument (104) and the master assembly (106) with the perception of telepresence.

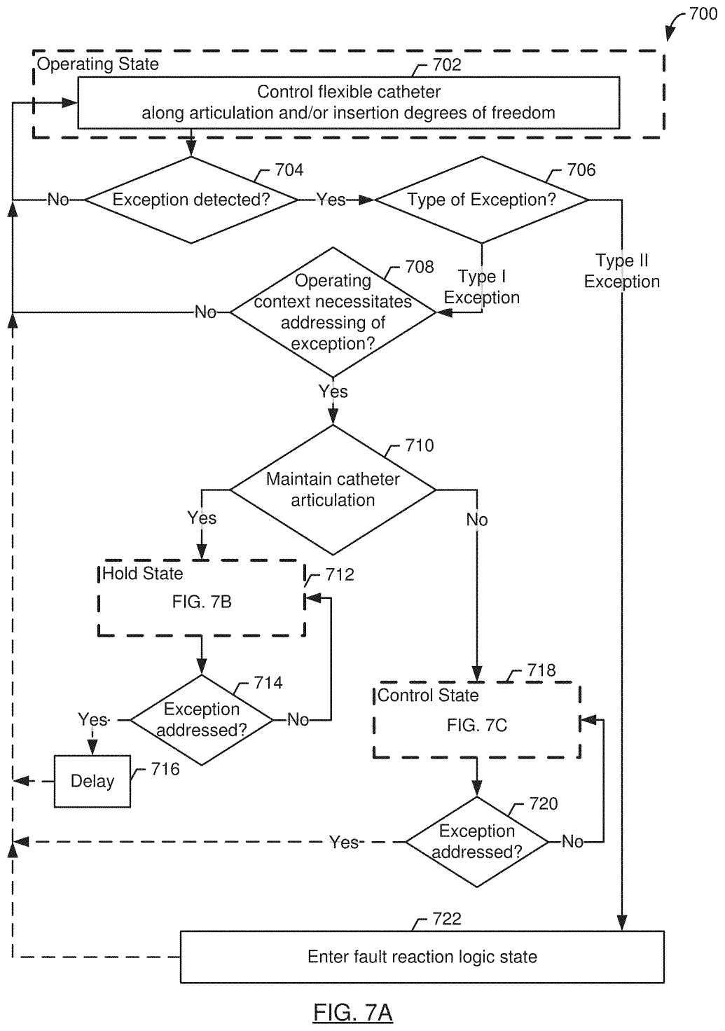

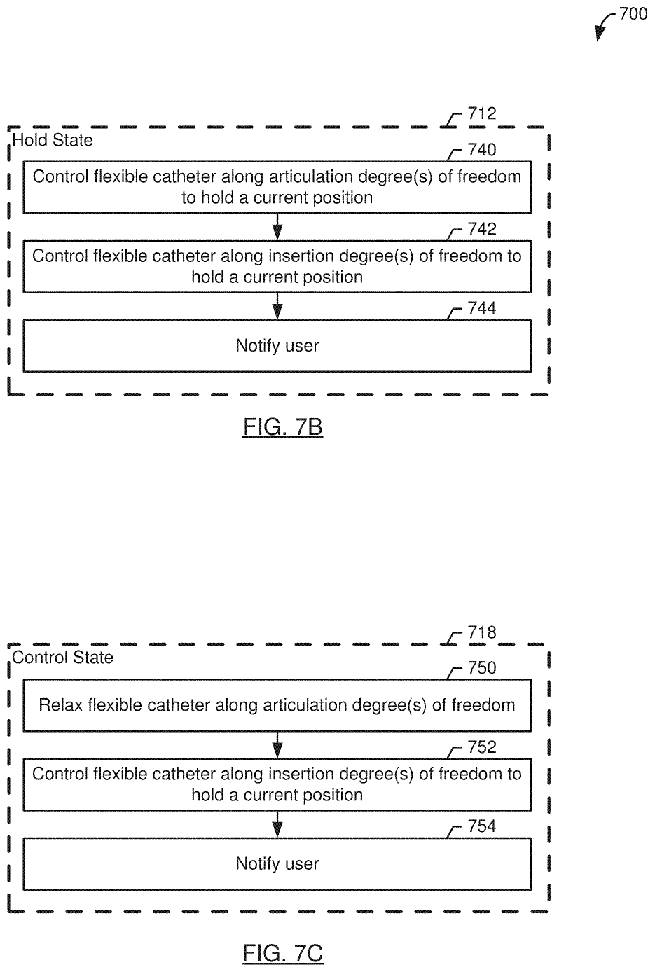

Continuing with FIG. 1A, the computer-assisted medical system (100) may also include a control system (112). The control system (112) includes at least one memory and at least one computer processor (not shown) for effecting control between the medical instrument (104), master assembly (106), sensor system (108), and display system (110). The control system (112) also includes programmed instructions (e.g., a non-transitory machine-readable medium storing the instructions) to implement some or all of the methods discussed below, including instructions for transitioning between states as described with reference to the flowchart of FIGS. 7A, 7B, and 7C. While the control system (112) is shown as a single block in the simplified schematic of FIG. 1A, the system may include multiple data processing circuits with one portion of the processing optionally being performed on or adjacent to the manipulator assembly (102), another portion of the processing being performed at master assembly (106), and/or the like. Any of a wide variety of centralized or distributed data processing architectures may be employed. Similarly, the programmed instructions may be implemented as a number of separate programs or subroutines, or they may be integrated into a number of other aspects of the systems described herein.

In one or more embodiments, the control system (112) may receive force and/or torque feedback from the medical instrument (104). Responsive to the feedback, the control system (112) may transmit signals to the master assembly (106). In some examples, the control system (112) may transmit signals instructing one or more actuators of manipulator assembly (102) to move the medical instrument (104) (e.g., to move a flexible catheter).

Any suitable conventional and/or specialized actuators may be used to actuate links or segments of the manipulator assembly (102) and/or the instrument (104). The one or more actuators may be separate from, or integrated with, manipulator assembly (102).

In one or more embodiments, the control system (112) may include a hierarchical control architecture, diagrammatically shown in FIG. 1B. The hierarchical control architecture may include a supervisor state machine (122), a mid-level controller (124) and a servo controller (126) for each of the actuators. The supervisor state machine (122) may be event-driven. Based on an event (130), the supervisor state machine (122) may send a behavioral primitive (132) to the mid-level controller (124). A behavioral primitive (132) may be a high-level command, specifying an overall goal, such as a target configuration of the manipulator assembly (102). In response, the mid-level controller (124) may issue servo commands (134) to the servo controller(s) (126). Each of the servo controllers (126) may generate a current command (136) to drive the actuators by a motor controller (128) (e.g., a brushless motor controller) generating the appropriate voltages (138) (e.g., pulse width modulated (PWM) voltages). More specifically, the mid-level controller (124) may perform numerical computations to configure, initialize and reset the servo controller(s) (126), based on the behavioral primitive (132). For example, the mid-level controller (124) may send parameters specifying positions and/or trajectories to the servo controllers (126). The servo controllers (126) may then drive the actuators, based on the commands received from the mid-level controller (124). The servo controllers (126) may report back to the supervisor state machine (122) by returning a servo state feedback (144), for example, to indicate whether movements have been successfully executed, according to the servo commands (134). Additional aspects of the control system (112) are described below, with reference to FIG. 7A, 7B, and 7C. In one or more embodiments, the supervisor state machine (122) and the mid-level controller (124) are low-latency. For example, a response to an event may be provided within 50 ms. Each of the servo controllers (126) may operate closed loop using a proportional integral derivative (PID), proportional derivative (PD), full state feedback, sliding mode, and/or various other control schemes. The feedback signal for a servo controller may be obtained from, for example, an encoder or resolver of the actuator being controlled by the servo controller (encoder feedback (140)). Alternatively, as further discussed below, a servo controller may also rely on other sensors for feedback. More specifically, a servo controller for controlling an articulation (e.g., bending) of a flexible catheter may use signals from an articulation sensor, e.g., a bend or shape sensor (shape sensor feedback signal (142)) to control the articulation or bending of the flexible catheter. A more detailed description of various aspects of the control system (112) is provided below with reference to FIG. 7A, 7B, and 7C.

Continuing with FIG. 1B, the control system (112) may operate in various modes, in addition to the above-described servo-control modes. For example, the control system (112) may control one or more of the joints of the manipulator assembly, described below, to float. A floating joint may be back-driven by an externally applied force without a control algorithm or a braking force counteracting sufficient externally applied force. For example, a user may apply a force meeting one or more criteria (e.g., for magnitude, direction, duration, frequency, etc.) to a link distal to the floating joint, causing the back-driving of the floating joint. A floating joint, in particular when floating in a degree of freedom affected by gravity (e.g., a vertical joint or in a non-horizontal direction), may further be gravity-compensated. In addition, a friction compensation may facilitate the back-driving. Additionally or alternatively, a floating joint may also be controlled to impose other characteristics such as a certain level of damping.

In one or more embodiments, floating joints may be particularly beneficial during a setup phase, allowing an assistant to manually position and/or orient the manipulator assembly (102) by back-driving the floating joints, during a setup phase in preparation for performing a procedure using the instrument (104). Multiple control modes may be combined during operation of the manipulator assembly, e.g., some joints may be position controlled to resist or rebound from external articulation of those joints, while other joints may be floating and facilitate external articulation of those other joints.

The control system (112) may optionally include a virtual visualization system to provide navigation assistance to the operator (O) when controlling the medical instrument (104) during an image-guided surgical procedure. Virtual navigation using the virtual visualization system may be based upon reference to an acquired preoperative or intraoperative dataset of anatomic passageways.

During a virtual navigation procedure, the sensor system (108) may be used to compute an approximate location of the medical instrument (104) with respect to the anatomy of the patient (P). The location can be used to produce both macro-level (external) tracking images of medical instrument (104) within the anatomy of the patient (P) and virtual internal images of the anatomy of the patient (P). The system may include one or more electromagnetic (EM) sensors, fiber optic sensors, and/or other sensors to register and display a medical instrument together with preoperatively recorded surgical images, such as those from a virtual visualization system. For example, PCT Publication WO 2016/191298 (published Dec. 1, 2016) (disclosing “Systems and Methods of Registration for Image Guided Surgery”), which is incorporated by reference herein in its entirety, discloses an example system.

The computer-assisted medical system (100) may further include optional operations and support systems (not shown) such as illumination systems, steering control systems, irrigation systems, and/or suction systems. In some embodiments, the computer-assisted medical system (100) may include more than one manipulator assembly and/or more than one master assembly. The exact number of teleoperational manipulator assemblies may depend on the surgical procedure and the space constraints within the operating room, among other factors.

In some embodiments, the manipulator assembly (102), the control system (112), the sensor system (108), and the display system (110) may all be supported by the support structure (114) or may be integrated into the support structure (114). Alternatively, one or more components (e.g., the manipulator assembly (102), the control system (112), the sensor system (108), and/or the display system (110)) may be mounted to the operating table (T) or integrated into the master assembly (106).

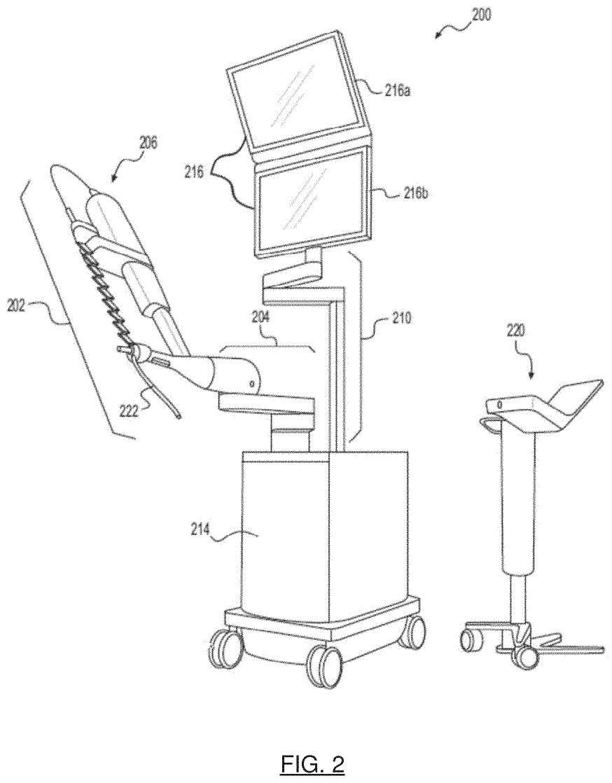

FIG. 2 illustrates, an example of a computer-assisted medical system (200), in accordance with one or more embodiments. The computer-assisted medical system (200) may include a master control (220) and a system cart (214) which supports a manipulator assembly (202) and a display system (216). The manipulator assembly (202) may be configured to support and position an elongate device such as the flexible catheter (222). Various elongate devices are described in PCT/US18/43041 (filed Jul. 20, 2018) (disclosing “Flexible Elongate Device Systems and Methods”), which is incorporated by reference herein in its entirety. The system cart (214) supports the display system (216) which includes the monitor support arm (210), and the display monitors (216a, 216b).

The computer-assisted medical system (200) of FIG. 2 also includes a master control (220) according to one or more embodiments, some aspects of which are discussed above with respect to master assembly (106). The master control (220) may include various input controls for an operator (e.g., the operator (O), FIG. 1A) to use for interactively controlling operations of the manipulator assembly (202), for example functions performed by the instrument manipulator (206). In one or more embodiments, the master control (220) includes a scroll wheel and a trackball on the surface, which allows the operator to control aspects of the computer-assisted medical system (200). In an example implementation, the scroll wheel may be rolled forwards or backwards in order to control the advancement/insertion or retraction of a medical instrument (e.g., the flexible catheter (222)) with respect to the patient anatomy, and the trackball may be rolled in various directions by an operator in order to steer the position of the distal end portion and/or distal tip of the flexible catheter (222), for example to control bend or articulation. Various systems and methods related to motion control consoles are described in PCT/US18/44419 (filed Jul. 30, 2018) (directed to “Systems and Methods for Safe Operation of a Device”) and U.S. patent application Ser. No. 16/049,640 (filed Jul. 30, 2018) (disclosing “Systems and Methods for Steerable Elongate Device”), which are incorporated by reference herein in their entireties.

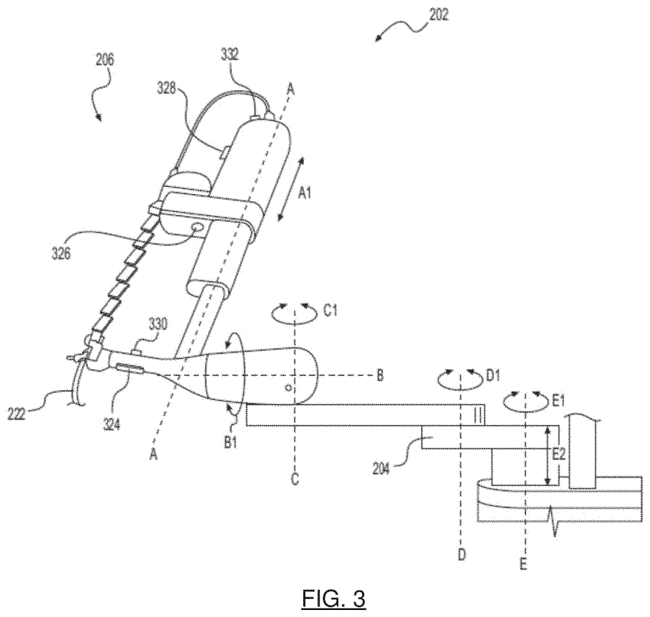

FIG. 3 shows the manipulator assembly (202) including an instrument manipulator (206) coupled to the support structure (204) in accordance with one or more embodiments of the invention. The links of the support structure (204) may include one or more nonservo controlled links (e.g., which may be manually positioned and locked into place) and/or one or more servo-controlled links (e.g., powered links that may be controlled in response to commands from a control system). The support structure (204) provides adjustments to position the instrument manipulator (206) at an optimal position and orientation and/or position the flexible catheter (222) to optimally position the flexible catheter (222) relative to the patient anatomy or other medical devices. For example, the support structure (204) may provide for a rotation (E1) about the axis (E), the extension/retraction (E2) along the axis (E), the rotation (D1) about the axis (D), and the rotation (C1) about the axis (C), and the rotation (B1), about the axis (B), to position the instrument manipulator (206) in a desired position relative to the table (T), medical devices, and/or the patient (P).

In some embodiments, optimal location and orientation can include alignment of the instrument manipulator (206) with respect to the patient anatomy, for example, for optimal positioning of the flexible catheter (222) to minimize friction of the flexible catheter (222) positioned within the patient anatomy (e.g. anatomical openings, patient vasculature, patient endoluminal passageways, etc.) or within medical devices coupled to patient anatomy (e.g., cannulas, trocars, endotracheal tubes (ETT), laryngeal esophageal masks (LMA), etc.). In other embodiments, optimal location and orientation of the instrument manipulator (206) may additionally or alternatively include optimizing the operator (O) ergonomics by providing sufficient operator workspace and/or ergonomic access to the flexible catheter (222) when utilizing various medical tools such as needles, graspers, scalpels, grippers, ablation probes, visualization probes, and/or the like, with the flexible catheter (222).

The instrument manipulator (206) may be further configured to provide teleoperational, robotic control, or other form of controlled translation or manual translation A1 along axis A to provide for insertion and retraction of the flexible catheter (222) with respect to the patient anatomy.

Each adjustment (e.g., A1, B1, C1, D1, E1, and E2) may be actuated by either robotic control or by manual intervention by an operator. For example, in one embodiment, each rotational or linear adjustment may be maintained in a stationary configuration using brakes such that depression of one or more buttons and switches releases one or more corresponding brakes allowing an operator to manually position the instrument manipulator. Additionally or alternatively, one or more adjustments may be controlled by one or more actuators (e.g., motors) such that an operator may use a button or switch to actuate a motor to alter the support structure (204) and/or the instrument manipulator (206) to position the manipulator assembly (202) in a desired configuration, typically to provide an optimal position and orientation of the instrument manipulator (206).

Continuing with FIG. 3, the manipulator assembly (202) may be equipped with various control buttons (324, 326, 328, 330) which may be used for various purposes such as an unlocking of the support structure (204) for free movement and adjustment of the coupled links to allow for adjustments C1, D1, E1, and/or E2 and/or linear adjustment A1 for manual translational movement by an operator rather than by robotic control to insert/retract a medical instrument (e.g., the flexible catheter (222)). In one or more embodiments, for safety purposes, the instrument manipulator (206) may only be manually movable in one direction along the linear axis A, such as retraction, and is not manually movable in the direction along the linear axis A that corresponds to insertion of the medical instrument, in order to prevent an operator from inadvertently or undesirably advancing the medical instrument with respect to the patient anatomy, which may result in harm to the patient. In another example, robotic or manual control of the rotational motion B1 about axis B may be enabled by depressing a switch. Further, one or more buttons may be used to control visual indicators, markers, and or images shown on monitors (216a, 216b), and/or a touchscreen on the master control (220).

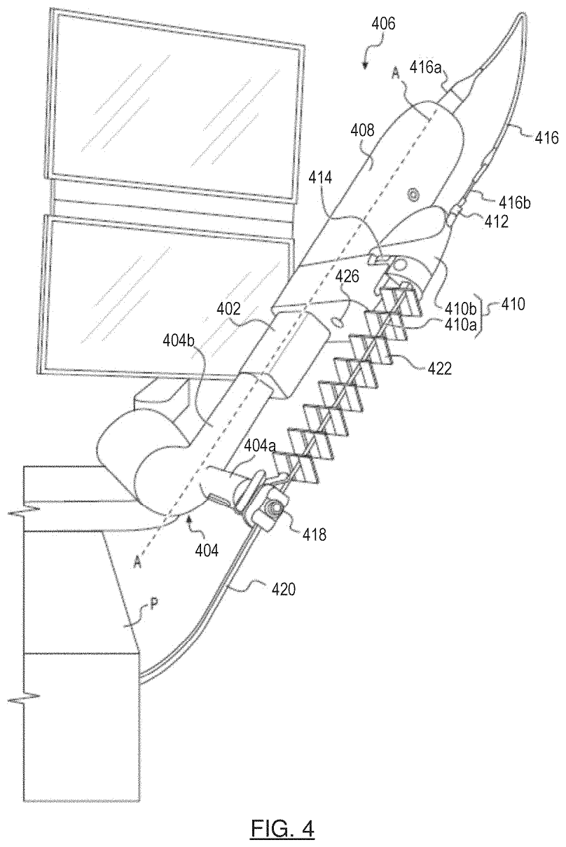

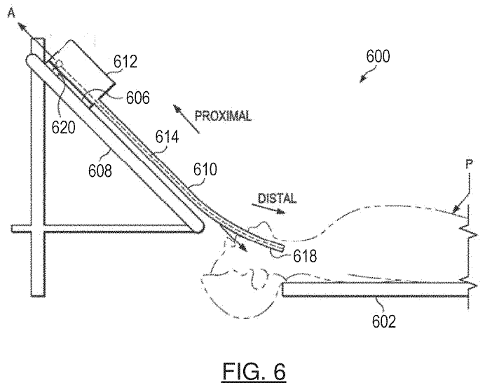

FIG. 4 shows an example of an instrument manipulator (406), which may be substantially similar to the instrument manipulator (206). The instrument manipulator (406) may include a base (404), an insertion stage (402), and an instrument carriage (408) to which a catheter assembly (410) is coupled. In one or more embodiments, the instrument manipulator (406) provides for insertion and retraction of the catheter assembly (410), with respect to the patient anatomy, by moving the instrument carriage (408) and insertion stage (402) in a telescoping manner relative to the base (404) and along the linear axis A, as further illustrated in FIG. 6. The instrument manipulator (406), thus, provides an insertion degree of freedom for the insertion and retraction of the flexible catheter (410a) along the linear axis A. In a medical scenario, the insertion may advance the flexible catheter (410a) into the patient anatomy, whereas the retraction may withdraw the flexible catheter (410a) from the patient anatomy.

The base (404) includes a shaft portion (404a) and a main portion (404b). As described in detail below, the shaft portion (404a) removably couples to a device connector or swivel connector (418) which receives the flexible catheter (410a). The insertion stage (402) is coupled to the main portion (404b) of the base (404) and translates along the main portion (404b). The instrument carriage (408) is coupled to and translates along the insertion stage (402). The catheter assembly (410) may include a flexible catheter (410a) and a control assembly (410b). The instrument carriage (408) couples to the control assembly (410b) at an instrument interface (414) of the instrument carriage (408). The instrument manipulator (406) also couples to a probe assembly (416) which includes a probe (416b) and a probe connector (416a). The probe assembly (416) may insert into a working lumen of the flexible catheter (410a) through the connector (412) on the control assembly (410b) and may run through the flexible catheter (410a). The probe (416b) may include, for example, a viewing scope assembly that provides images of a surgical site. The instrument carriage (408) may include electronic and optical components providing probe (416b) with endoscopic capabilities. In some embodiments, the probe assembly (416) may be detached from the instrument manipulator (406) and flexible catheter control assembly (410b), and removed from the catheter assembly (410). Alternative instruments such as biopsy needles, ablation tools, and other flexible instruments may be coupled to the instrument manipulator (406) and/or the catheter assembly (410), through the flexible catheter (410a) working lumen.

Continuing with FIG. 4, the device connector or swivel connector (418) may include a manipulator interface which may be removably coupled to the base (404), a distal end which may be removably coupled to a patient medical device (420), e.g., an endotracheal tube, and a proximal end which may receive the flexible catheter (410a). The patient medical device (420) (e.g., an endotracheal tube, a laryngeal mask airway, a cannula, etc.) may be fixed to the patient anatomy to facilitate insertion of various medical devices into the patient anatomy. For example, the patient medical device (420) may be an endotracheal tube inserted into the mouth and trachea of the patient (P) to provide a conduit for the flexible catheter (410a) to be navigated within the lungs of the patient (P) to facilitate imaging, biopsy, and/or treatment. Various systems and methods related to device connectors are described in PCT/US2018/017085 (filed Feb. 6, 2018) (disclosing “Systems and Methods for Coupling Components of a Medical System”), which is incorporated by reference herein in its entirety. In some embodiments, the flexible catheter (410a) runs through a catheter guide (422), which is a selectively collapsible and extendable device that supports the length of the flexible catheter (410a) during movement of the instrument carriage (408). The flexible catheter (410a) without guidance may buckle in regions with no lateral support, e.g., in the space between the instrument interface (414) and the device connector (418). To avoid the buckling, the catheter guide (422) may operate as an anti-buckling guide by providing lateral support to the flexible catheter (410a). Various systems and methods related to catheter guides are described in PCT/US2017/041160 (filed Jul. 7, 2017) (disclosing “Guide Apparatus for Delivery of an Elongate Device and Methods of Use”), which is incorporated by reference herein in its entirety.

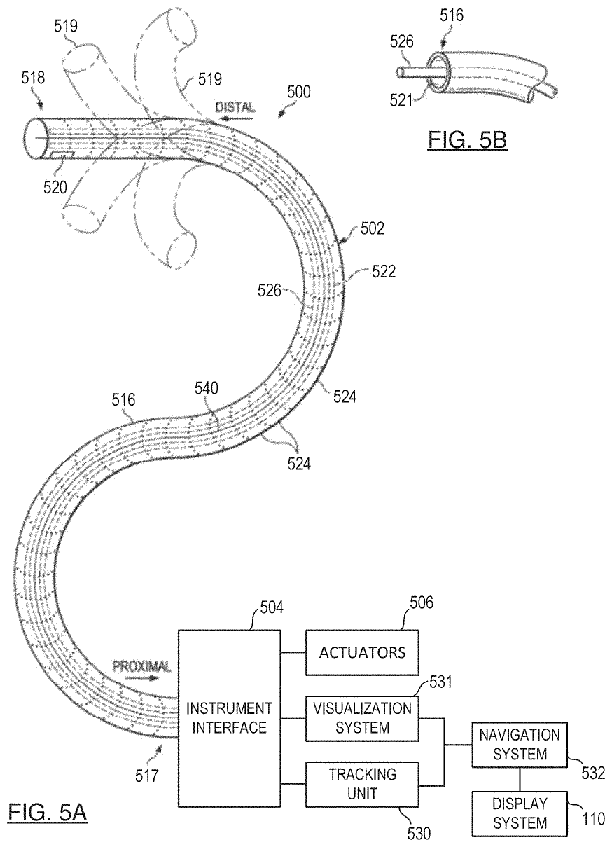

FIG. 5A is a simplified diagram of a computer-assisted medical system (500) in accordance with one or more embodiments. While the previously discussed figures primarily illustrate aspects of the overall computer assisted medical system (100) and the instrument manipulator (206), FIG. 5A and FIG. 5B primarily illustrate aspects related to the flexible catheter (502). The computer-assisted medical system (500) may be similar to the computer-assisted medical system (100) previously introduced with reference to FIG. 1A. The computer-assisted medical system (500) may include the flexible catheter (502) (or more generally, an elongate flexible device) on an instrument interface (504). The instrument interface may be the physical interface that enables the coupling of the flexible catheter to other components of the computer-assisted medical system (500). In the example of FIG. 4, the instrument interface may be part of the instrument carriage (408).

In one or more embodiments, the flexible catheter (502) includes a flexible body (516) having a proximal portion (517) and a distal portion (518) (e.g., a tip portion). The flexible body (516) may have an outer diameter of approximately 3 mm. Other flexible body outer diameters may be larger or smaller.

The flexible body (516) may include a lumen or channel (521) sized and shaped to receive a medical instrument (526), as shown in FIG. 5B. FIG. 5B is a simplified diagram of the flexible body (516) with the medical instrument (526) extended, in accordance with one or more embodiments. The medical instrument (526) may be used for procedures including but not limited to surgery, biopsy, ablation, illumination, irrigation, and/or suction. For example, the medical instrument (526) may be a flexible bronchial instrument, such as a bronchoscope or bronchial catheter, for use in examination, diagnosis, biopsy, or treatment of a lung. The medical instrument (526) may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The medical instrument (526) may be deployed through the lumen (521) of the flexible body (516) and may be used at a target location within the anatomy. The medical instrument (526) may include, for example, image capture probes, biopsy instruments, laser ablation fibers, and/or other surgical, diagnostic, or therapeutic tools. Medical tools may include end effectors having a single working member such as a scalpel, a blunt blade, an optical fiber, an electrode, and/or the like. Other end effectors may include, for example, forceps, graspers, scissors, clip appliers, and/or the like. Other end effectors may further include electrically activated end effectors such as electrosurgical electrodes, transducers, sensors, and/or the like. In one or more embodiments, the medical instrument (526) is a biopsy instrument which may be used to remove sample tissue or a sampling of cells from a target anatomic location. The medical instrument (526) may be used with an image capture probe also within the flexible body (516).

Returning to FIG. 5A, the medical instrument (526) may be an image capture probe that includes a distal portion with a stereoscopic or monoscopic camera at or near the distal portion (218) of the flexible body (516) for capturing images (including video images) that are processed by a visualization system (531) for display and/or provided to the tracking unit (530) to support tracking of the distal portion (518) and/or one or more of the segments (524). The image capture probe may include a cable coupled to the camera for transmitting the captured image data. In some examples, the image capture instrument may be a fiber-optic bundle, such as a fiberscope, that couples to the visualization system (531). Alternatively, the medical instrument (526) may itself be the image capture probe. The medical instrument (526) may be advanced from the opening of the lumen (521) to perform the procedure and then retracted back into the lumen when the procedure is complete. The medical instrument (526) may be removed from the proximal portion (517) of the flexible body (516) or from another optional instrument port (not shown) along the flexible body (516).

In one or more embodiments, one or more actuators (506) may be used to drive the flexible catheter (502). For example, one actuator may be used to drive the catheter along the insertion degree of freedom of the flexible catheter. Additional actuators may be used to drive the catheter along one or more articulation degrees of freedom, as further discussed below. In one or more embodiments, the flexible body (516) houses pull-wires, linkages, or other steering controls (540) that extend between the actuators (506) and the distal portion (518) to controllably bend the distal portion (518) as shown, for example, by the broken dashed line depictions (519) of the distal end (518). In one or more embodiments, at least four pull-wires (540), actuated by at least four actuators (506) are used to provide independent up-down steering to control a pitch of the distal portion (518) and left-right steering to control a yaw of the distal portion (518). Steerable elongate devices are described in detail in U.S. patent application Ser. No. 13/274,208 (filed Oct. 14, 2011) (disclosing “Catheter with Removable Vision Probe”), which is incorporated by reference herein in its entirety. More details on control mechanisms for control systems for controlling flexible catheters are provided in U.S. patent application Ser. No. 62/671,758 (disclosing “Control Mechanism of a Catheter Control System”), which is incorporated by reference herein in its entirety. In one or more embodiments, the actuators (506) may removably couple to the flexible catheter (502) via the instrument interface (504). The actuators (506) may be, for example, servo motors, hydraulic and/or pneumatic actuators, etc. Referring to FIG. 4, the actuators (506) may be housed in, for example, the control assembly (410b) of the instrument carriage (408).

Continuing with FIG. 5A, when an actuator applies torque, a capstan (not shown) attached to the drive shaft of the actuator may be rotated. The rotation may cause a further wrapping or unwrapping of the pull-wire (540) around the capstan, thereby actuating the pull-wire to steer the distal end portion (518) of the flexible catheter (502). Each pull-wire (540) may be driven by a separate actuator. Accordingly, the tension on each of the pull-wires (540) may be individually controlled.

Monitoring a rotation position (e.g., angle) and/or rotational velocity of the capstan or actuator may be used to provide an indication of how far the pull-wire (540) is being released or pulled. Thus, the rotation angle and/or rotational velocity of the capstan and/or the torque applied by the actuator to drive the capstan may provide useful feedback on the steering to be applied at the distal portion (518). Which way the distal portion (518) bends may depend upon the placement of the pull-wire (540) with respect to other pull-wires that are also contributing to steering. Actuator sensors such as resolvers, encoders, potentiometers, and other mechanisms may be used to track the rotation and/or orientation of capstans and/or actuators. The motor currents of the actuators may also be used to calculate forces and/or torques being applied to the pull-wires (540).

When configured to operate as antagonists, pairs of actuators (506) (e.g., one pair of actuators for pitch control of the distal portion (518) and one pair of actuators for yaw control of the distal portion (518)), may be used to articulate the distal portion (518) and control a stiffness of the flexible body (516). Further, by maintaining a minimum level of tension on the pull-wires (540), slack in the pull-wires (540) may be avoided. Releasing or reducing the force on the pull-wires (540) of the flexible catheter (502) may cause a corresponding reduced stiffness or rigidity in the flexible catheter (502). Similarly, applying or increasing a pulling force in the pull-wires (540) of the flexible body (516) may cause an increase in stiffness or rigidity of the flexible catheter (502). For example, the flexible body (516) may become stiffer with multiple steering pull-wires being pulled concurrently. The stiffness or rigidity of the flexible catheter (502) may be a closed-loop stiffness or rigidity controlled by the control system. Examples of a closed-loop catheter control system and methods are described, for example, in U.S. patent application Ser. No. 13/274,198 (filed Oct. 14, 2011) (disclosing “Catheters with Control Modes for Interchangeable Probes”) which is incorporated by reference herein in its entirety.

In one or more embodiments, the computer-assisted medical system (500) may include a tracking unit (530) for determining the position, orientation, speed, velocity, pose, and/or shape of the distal portion (518) and/or of one or more segments (524) along the flexible body (516), using one or more sensors and/or imaging devices as described in further detail below. The tracking unit (530) may be implemented as hardware, firmware, software or a combination thereof. One or more aspects of the tracking unit may be performed by the processor(s) of the control system (112) in FIG. 1A and FIG. 1B.

The tracking unit (530) may track the distal portion (518) and/or one or more of the segments (524) using an articulation sensor such as the shape sensor (522). The shape sensor (522) may include an optical fiber aligned with the flexible body (516) (e.g., provided within an interior channel (not shown) of the flexible body (516) or mounted externally). In one embodiment, the optical fiber has a diameter of approximately 200 μm. The diameter of the fiber may be larger or smaller, without departing from the disclosure. The optical fiber of the shape sensor (522) forms a fiber optic bend sensor for determining the shape of flexible body (516). Optical fibers including Fiber Bragg Gratings (FBGs) may be used to provide strain measurements in structures in one or more dimensions. Various systems and methods for monitoring the shape and relative position of an optical fiber in three dimensions are described in U.S. patent application Ser. No. 11/180,389 (filed Jul. 13, 2005) (disclosing “Fiber optic position and shape sensing device and method relating thereto”); U.S. patent application Ser. No. 12/047,056 (filed on Jul. 16, 2004) (disclosing “Fiber-optic shape and relative position sensing”); and U.S. Pat. No. 6,389,187 (filed on Jun. 17, 1998) (disclosing “Optical Fibre Bend Sensor”), which are all incorporated by reference herein in their entireties. Other suitable strain sensing techniques, such as Rayleigh scattering, Raman scattering, Brillouin scattering, and Fluorescence scattering may be employed, without departing from the disclosure. Alternatively, the shape of the elongate device may be determined using other techniques. For example, a history of the distal end pose of flexible body (516) may be used to reconstruct the shape of flexible body (516) over an interval of time.

Continuing with FIG. 5A, the tracking unit (530) may receive a signal from the shape sensor (522). The tracking unit (530), in one or more embodiments, processes the signal to obtain information about the shape of the flexible body (516). The obtained information may be for the shape of the distal portion (518) and/or for one or more segments (524) along the flexible body (516). The tracking unit (530), in combination with the shape sensor (522) may provide feedback on, for example, an articulation of the flexible catheter (502). The tracking unit may further be configured to detect abnormality of the shape sensor (522) and/or the tracking unit itself. For example, the tracking unit (530) may detect noise (such as vibrations) in the raw signal. The tracking unit (530) may further detect a poor raw signal associated with a contaminated or poorly fitted fiber optic connector to the shape sensor (522).

In some embodiments, the tracking unit (530) optionally and/or additionally tracks the distal portion (518) using a position sensor system (520). The position sensor system (520) may use any appropriate sensing technology or combination of sensing technologies, such as, for example, electromagnetic techniques. An electromagnetic (EM) sensor system may include one or more conductive coils that may be subjected to an externally generated electromagnetic field. Each coil of such an EM sensor system used to implement position sensor system (520) then produces an induced electrical signal having characteristics that depend on the position and orientation of the coil relative to the externally generated electromagnetic field. In some embodiments, the position sensor system (520) may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point. Further description of a position sensor system is provided in U.S. Pat. No. 6,380,732 (filed Aug. 11, 1999) (disclosing “Six- Degree of Freedom Tracking System Having a Passive Transponder on the Object Being Tracked”), which is incorporated by reference herein in its entirety. The position sensor system (520) may be used as an articulation sensor. For example, a plurality of sensors (520), such as EM sensors, may be positioned along the flexible catheter 502 (e.g., at a plurality of segments 524), and articulation (e.g., shape) of the flexible catheter 502 may be determined based on detected positions of the sensors (520).

The information from the tracking unit (530) may be sent to a navigation system (532) where it may be combined with information from the visualization system (531) and/or the preoperatively obtained models to provide the physician or other operator with real-time position information. The real-time position information may be displayed on display system 110 of FIG. 1A for use in the control of the computer-assisted medical system (500). In some examples, the control system (112) of FIGS. 1A and 1B may utilize the position information as feedback for positioning the computer-assisted medical system (500). Various systems for using fiber optic sensors to register and display a surgical instrument with surgical images are provided in U.S. patent application Ser. No. 13/107,562, filed May 13, 2011, disclosing, “Medical System Providing Dynamic Registration of a Model of an Anatomic Structure for Image-Guided Surgery,” which is incorporated by reference herein in its entirety.

FIG. 6 schematically shows a medical scenario including a side view of a patient coordinate space, in accordance with one or more embodiments. The medical scenario (600) may include a patient (P) positioned on a platform (602). The instrument carriage (606) is mounted to an insertion stage (608) as also shown in FIG. 4. The instrument carriage (606) may be used to control insertion/retraction of the flexible catheter (610) (e.g., motion along the insertion axis A) into/out of the anatomy of the patient (P), thereby establishing an insertion degree of freedom for the flexible catheter (610). The instrument carriage (606) may further be used to control motion (e.g., articulation) of the distal portion (618) of the flexible catheter in multiple directions including yaw and pitch. The instrument carriage (606) or insertion stage (608) may include an actuator, such as a servomotor, (not shown) that controls motion of the instrument carriage (606) along the insertion stage (608). Further, the instrument carriage (606) may include actuators that control the articulation of the distal portion (618) of the flexible catheter.

The flexible catheter (610) may be coupled to an instrument interface (612), previously described for FIG. 4 and FIG. 5. The instrument interface (612) may be coupled and fixed relative to the instrument carriage (606). In one or more embodiments, an optical fiber shape sensor (614) provides information about the configuration of the flexible catheter (610), including, for example, position and/or orientation of the distal portion (618) of the catheter (610). A position measuring device (620) may provide information about the position of the instrument interface (612) as it moves on the insertion stage (608) along the retraction and/or insertion axis A (such as a direction along the longitudinal central axis of the instrument body) establishing the insertion degree of freedom of the flexible catheter (610). The position measuring device (620) may include resolvers, encoders, potentiometers, and/or other sensors that determine the rotation and orientation of drive shafts controlling the motion of the instrument carriage (606) and consequently the motion of the instrument interface (612). The insertion stage (608) may be linear, curved, or a combination thereof.

While FIG. 1A, FIG. 1B, FIG. 2, FIG. 3, FIG. 4, FIG. 5A, FIG. 5B, and FIG. 6 show various configurations of components, other configurations may be used without departing from the scope of the disclosure. For example, various components may be combined to create a single component. As another example, the functionality performed by a single component may be performed by two or more components. Further, while the components are described in context of medical scenarios, embodiments of the disclosure may be equally applicable to other domains that involve robotic manipulation, e.g., non-medical scenarios or systems. Embodiments of the disclosure may be suitable for use in, for example, surgical, diagnostic, therapeutic, and/or biopsy procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems and general robotic or teleoperational systems. Rather than interacting with an anatomy of a patient or subject, the systems, instruments and methods may operate in or interact with a worksite of any type.

FIGS. 7A, 7B, and 7C show a flowchart in accordance with one or more embodiments. One or more of the operations in FIGS. 7A, 7B, and 7C may be performed by various components of the systems, previously described with reference to FIG. 1A, FIG. 1B, FIG. 2, FIG. 3, FIG. 4, FIG. 5A, FIG. 5B, and FIG. 6. These figures describe particular manipulator assemblies and particular flexible catheters, the manipulator assemblies and flexible catheters having certain degrees of freedom. However, the subsequently described methods are not limited to a particular configuration of manipulator assemblies, flexible catheters and/or degrees of freedom. Instead, the methods are applicable to any type of flexible catheter supported by a manipulator assembly, used in any type of scenario.

While the various operations in these flowcharts are presented and described sequentially, one of ordinary skill will appreciate that some or all of the operations may be executed in different orders, may be combined or omitted, and some or all of the operations may be executed in parallel. Additional operations not shown in the flowchart may also be performed. Furthermore, the operations may be performed actively or passively. For example, some operations may be performed using polling or be interrupt driven in accordance with one or more embodiments of the disclosure. By way of an example, determination operations might not require a processor to process an instruction unless an interrupt is received to signify that condition exists in accordance with one or more embodiments of the disclosure. As another example, determination operations may be performed by performing a test, such as checking a data value to test whether the value is consistent with the tested condition in accordance with one or more embodiments of the disclosure. Accordingly, the scope of the disclosure should not be considered limited to the specific arrangement of operations shown in FIGS. 7A, 7B, and 7C.

The flowchart of FIGS. 7A, 7B, and 7C shows an example method (700) for transitioning between states (e.g., operating states, control states, etc.) in flexible robotic-assisted devices, in accordance with one or more embodiments. For example, the methods monitor a computer-assisted medical system for exceptions. The computer-assisted medical system, in one or more embodiments, may be equipped with a flexible catheter. In a normal operating state, various aspects of the flexible catheter, such as insertion and retraction movements along an insertion degree of freedom and/or articulation of the flexible catheter along one or more articulation degrees of freedom (e.g., pitch and/or yaw), may be servo-controlled. In the normal operating state, movement of the flexible catheter may be under control (e.g., teleoperational control) of an operator, assistant, an autonomous control algorithm, etc. When an exception is detected, the system may switch from the normal operating state to another state, such as a hold state, a control state, or a fault reaction logic state.

In the hold state, various degrees of freedom, including the articulation degrees of freedom and the insertion degree of freedom may be controlled to hold a current position. The holding of the current position may be beneficial when certain operational exceptions occur. Consider, for example, a scenario in which one or more input devices (e.g., a trackball and/or scroll wheel) is used to control the articulation degrees of freedom and the insertion degrees of freedom. The input device(s) may get contaminated, for example, by saline or other fluids that may be present in medical scenarios. The contamination may interfere with the function of the input device(s). Accordingly, a cleaning of the input device(s) may be necessary to address the contamination. For example, the master control may include a removable trackball and/or scroll wheel, and cleaning may involve removal of the trackball and/or scroll wheel from the master control, cleaning of the trackball and/or scroll wheel, and reinsertion of the cleaned trackball and/or scroll wheel. During these operations, trackball and/or scroll wheel signals may be absent and/or inaccurate. To avoid unintentional movement along the articulation degrees of freedom and/or the insertion degree of freedom, the trackball signals and/or the scroll wheel signals may, thus, need to be ignored during the removal, cleaning, and re-insertion of the trackball and/or scroll wheel. The hold state may further be beneficial when other types of operational exceptions occur, for example during a signal loss from a sensing probe mounted on the distal portion of the flexible catheter, or during other malfunctions or disruptions that are not directly associated with tracking the motion of the flexible catheter.

In the control state, one or more degrees of freedom, such as articulation degrees of freedom, may be relaxed. For example, rather than controlling an articulation based on a commanded articulation, a portion (e.g., a distal, steerable portion) or a length of a flexible body of the flexible catheter may relax. When relaxed, an external force acting on the relaxed portion may cause motion of the relaxed portion when the relaxed portion yields to the external force. For example, in a medical scenario, a surrounding tissue pressing against the relaxed portion may cause the relaxed portion to bend in response to the surrounding tissue pressing against the relaxed portion. The ability of a flexible catheter in a relaxed or limp state to bend in response to external forces may be beneficial when operating in a delicate environment such as during an interventional procedure.

In the presence of an exception, some degree of uncertainty may be introduced. For example, a malfunctioning shape sensor might not provide reliable data about the current pose of the flexible catheter's distal portion. As a result of the uncertainty, it may be difficult to issue a proper control command for driving the flexible catheter, as discussed in detail below. To address the uncertainty caused by the exception, the actuators for the articulation degree of freedom may be provided with command signals to relax the flexible body or steerable portion of the flexible body. In a relaxed state, the flexible body may be less likely to cause damage to surrounding anatomy. For example, the flexible catheter may be withdrawn without causing damage when the distal portion is in a relaxed state because, during a withdrawal, the limp distal portion may passively follow the surrounding anatomy rather than exerting a force on the surrounding anatomy as may occur under servo control, e.g., when a particular degree of articulation is commanded.

The subsequently described operations are for methods for transitioning between an operating state, a hold state, a control state, and/or a locked state. Different types of exceptions may cause different behaviors, as described below.

In operation 702, an articulation degree of freedom of the flexible catheter is controlled (e.g., servo-controlled). Further, an insertion degree of the flexible catheter may be controlled (e.g., servo-controlled). The execution of operation 702 may occur during a normal operating state of the computer-assisted medical system, such as when no exceptions are detected. The actuators (e.g., servo motors, stepper motors, linear motors, direct drive motors, and/or the like) associated with the insertion and articulation degrees of freedom may be energized. If the actuator associated with the insertion degree of freedom includes a brake, the brake may be released.

When in the normal operating state, the servo-control may be used to operate in various sub-states, including a driving mode, a slow passive mode, and/or a full passive mode.

In the driving mode, user inputs provided via the input interface may result in corresponding movements of the flexible catheter. The driving mode may also include various modes of operation, such as a parking mode, retraction mode, and insertion mode. Various modes of operation, including going limp during a retraction of the flexible catheter are described in PCT Patent Application No. PCT/US2019/053953, filed Oct. 1, 2019, disclosing, “Systems and Methods for Control of Steerable Devices,” which is incorporated by reference herein in its entirety. The rigidity or stiffness of the catheter may vary depending on the mode of operation. Various aspects of stiffness profiles are described in U.S. patent application Ser. No. 16/311,514, filed May 30, 2017, disclosing, “Systems and Methods for Flexible Computer-Assisted Instrument Control,” which is incorporated by reference herein in its entirety. The driving mode may enable a user to operate the system, or components of the system such as the flexible catheter along the insertion degree of freedom and/or along the articulation degree(s) of freedom.

In the full passive and slow passive modes, the rigidity of the catheter may be gradually reduced according to various rigidity profiles. The full passive and slow passive modes may be selected by the operator, such as by pressing a button on the input device. The full passive mode may be used when retracting the catheter from the anatomy, whereas the slow passive mode may be used for slight adjustments in the catheter position which require retraction.

When under servo-control (e.g., enabling the driving, full passive and/or slow passive modes), a controller may compare a sensor signal obtained from the flexible catheter and a commanded signal (e.g., the user input, an automated control command, etc.) to obtain an error used to drive the actuator(s) associated with the degree(s) of freedom to be controlled. To control the flexible catheter along an articulation degree of freedom, a pair of actuators may be used. These actuators may operate in an antagonistic manner, with one actuator increasing tension on a pull-wire (e.g., tightening) for the articulation degree of freedom and the other actuator decreasing tension on a pull-wire (e.g., loosening) for the same articulation degree of freedom. To control pitch and yaw degrees of freedom of the flexible catheter, two pairs of actuators may be employed. By independently controlling the two actuators associated with a degree of freedom, the stiffness or rigidity of the flexible catheter, in the degree of freedom may be controlled: The stiffness may increase when both actuators increase tension on the respective pull-wires, and the stiffness may decrease when both actuators decrease tension on the respective pull-wires. The sensor signal(s) used for controlling the actuator(s) may be obtained from a shape sensor (e.g., a fiber shape sensor, EM sensor, and/or the like), as previously described. Accordingly, when under control, the sensed actual articulation (e.g., of a distal portion) may be used for feedback, rather than relying on actuator sensor signals (e.g., from encoders) exclusively. In other words, in one embodiment, distal feedback based on distal sensor signals (measured at the articulating portion of the flexible catheter) are used to implement a closed loop control paradigm, rather than using encoder signals of the actuators to control the articulating portion in an open loop manner. Depending on the design of the flexible catheter, the control of the articulation degree(s) of freedom may be for a distal portion of the flexible catheter, and/or for multiple segments along the flexible catheter.

To control the flexible catheter along the insertion degree of freedom, a sensor signal, (e.g., an encoder signal, an insertion sensor, etc.) may be relied upon. The controlled actuator may drive the instrument carriage, which in turn may result in an insertion or retraction of the flexible catheter. If the actuator is equipped with a brake, the brake may be released during controlled operation.

During the normal operating state, the system may determine whether an exception has been detected, and the system may switch to another state if an exception is detected. Returning to FIG. 7A, in operation 704, a test may be performed to detect an exception. Exceptions may be detected by the previously described supervisor state machine in an event-driven manner, for example, based on a flag that has changed. The detection may be near instantaneous and may take, for example, 50 ms or less, or the detection may be over a period of time. The supervisor state machine may monitor for many different types of exceptions. Some of these exceptions may be actual system errors, whereas other exceptions may be user-induced, e.g., caused by an intentional user input, an erroneous action by a user, etc. Some of these exceptions are subsequently described:
(i) Distal sensor data exception: For various reasons, the distal sensor data (e.g., obtained from a bend or shape sensor) might not be sufficiently accurate to allow servo-control of the actuator(s) associated with the articulation degree(s) of freedom of the flexible catheter. The tracking unit receiving the raw data from the shape sensor may detect the flawed shape sensor data and may notify the supervisor state machine using one or more flags. For example, a shape sensor may be equipped with redundant sensing modalities and if a mismatch beyond a certain degree is detected, an exception may be triggered. Further, an exception may be triggered if data samples collected over time are noisy or include oscillations, if consecutive data samples are different beyond a threshold (which may be set based on how much the flexible catheter can realistically move during a set time interval), if data samples indicate a configuration of the flexible shape sensor (e.g., fiber shape sensor) that is physically unrealistic, etc. Any such exception may result in the issuance of a low confidence in shape sensor data flag. An exception may be triggered when a single bad sensor data reading is detected or when sensor data readings are repeatedly bad. Various causes for shape sensor data exceptions exist. These causes include, but are not limited to, physical vibrations of the flexible catheter causing vibrations of the shape sensor, an unreliable connection to the shape sensor (e.g., as a result of a dirty fiber-optic connector), etc.
(ii) Excessive articulation actuator travel exception: An actuator associated with an articulation degree of freedom of the flexible catheter may be expected to have a range of positions and/or velocities within specified boundaries. If these boundaries are exceeded, an excessive articulation actuator travel exception may be triggered.
(iii) Lack of consistency between proximal and distal sensing exception: A set amount of movement of an actuator may be expected to result in a set amount of articulation of the articulating portion of the flexible catheter, based on the mechanical coupling of the actuator to the articulating portion using a pull-wire. If a deviation of positions and/or velocities of the proximal sensing (performed, e.g., by an encoder) and the distal sensing (performed, e.g., by the bend or shape sensor) beyond a set threshold is detected, an exception may be triggered.
(iv) Input device missing exception: If an input device, e.g., a trackball or scroll wheel is not detected, an exception may be triggered. The input device missing exception may occur, for example, when the trackball and/or scroll wheel is removed for cleaning and may persist until the trackball and/or scroll wheel is reinserted. The absence of the trackball and/or scroll wheel may be detected using, for example, an optical, capacitive, or mechanical sensor.
(v) Manipulator assembly joint exception: One or more of the joints of the manipulator assembly, termed setup joints, may be used during a setup phase, e.g., to position and/or orient the instrument manipulator prior to performing an actual procedure. During the procedure, the setup joints may be locked in position using brakes to provide a stable base for the operation of the instrument. An exception may be triggered when a brake is found to be released, when a setup joint is found to be slipping, etc. Setup joint slippage may be detected by an encoder or other joint sensor tracking joint position. Similarly, if the manipulator assembly is installed on a cart that is moveable on wheels, the status of the wheel brakes and/or wheel movement may be monitored, and movement of the cart, or brakes that are not engaged, may trigger an exception.
(vi) Catheter guide exception: The state of the catheter guide may be monitored.

As previously explained, the catheter guide may provide lateral support for a flexible catheter. For example and with reference to FIG. 4, the catheter guide (422) may be attached to the base (404) of the instrument manipulator (406) to provide lateral support for the flexible catheter (410a). A catheter guide that is found to be missing or not properly attached (e.g., to the base 404), may trigger a catheter guide exception.
(vii) Swivel connector undocked exception: As previously explained, the swivel connector or device connector (418) may be removably docked to the base (404) and may be configured to receive the flexible catheter (410a). The undocking of the swivel connector (418) may trigger an exception.

The above events have in common that they might not be caused by a failure of an actuator associated with an articulation degree of freedom of the flexible catheter. In other words, the one or more actuators associated with the articulation degree(s) of freedom may still be controllable, and no other event that would render the system dysfunctional is present.
(viii) Articulation actuator failure exception: The actuator(s) associated with the articulation degree(s) of freedom may produce exceptions for various reasons. For example, an actuator may be determined to be defective if the resistance of a motor winding deviates from an expected resistance. A defective actuator may, thus, trigger an articulation actuator failure exception.
(ix) Pull-wire tension exception: If the pull-wire tension drops below a specified limit corresponding to a minimum pull-wire tension, which could cause the pull-wire to derail, an exception may be triggered.
(x) Input device unreliable exception: An input device, e.g., a scroll wheel or a trackball, may be equipped with redundant sensors for reliability. If the redundant sensors provide different values beyond a set threshold, and for a duration exceeding a specified length of time, an exception may be triggered.
(xi) Distal sensor failure exception: The shape sensor may provide error status bits indicating a failure (e.g., beyond inaccuracy). For example, an error status bit may indicate that the shape sensor or the shape sensor readout interface is defective. The failure of the shape sensor or a component of the shape sensor may trigger a distal sensor failure exception.
(xii) Manipulator assembly exception: If a voltage or current of an actuator exceeds a threshold, if a difference between primary and secondary sensors (e.g., incremental sensors on actuators and additional rotational or linear sensors measuring at the link being actuated) exceeds a threshold, an exception may be triggered. Similarly, if an excessive force or torque, an excessive commanded velocity and/or acceleration, a significant difference between a commanded and an actual joint position, the release of a brake for longer than a specified time interval, is detected, an exception may be triggered.
(xiii) System errors: An exception may also be triggered by invalid message exchanges between the supervisor state machine, the mid-level controller and the servo controllers.
(xiv) Additionally, external commands, such as a user request, may trigger an exception.

The above events have in common that they may have the potential to impair the control of the articulation degree(s) of freedom.

Those skilled in the art will appreciate that the described examples are to be understood as non-limiting examples—many other events may exist.

Returning to FIG. 7A, if no exception is detected (operation 704: no), the method may continue with the execution of operation 702. If an exception is detected (operation 704: yes), the execution of the method may proceed with operation 706.

In operation 706, the exception may be classified based on a type of exception. For example, the exception may be a type I exception or a type II exception. For type I exceptions, the execution of the method may proceed with operation 708. For type II exceptions, the execution of the method may proceed with operation 722. Type I exceptions may be associated with events under which the one or more actuators associated with the articulation degree(s) of freedom of the flexible catheter are still controllable, and where no other event that would render the system dysfunctional is present. In contrast, type II exceptions may be associated with events that limit or prevent controllability of the one or more actuators associated with the articulation degree(s) of freedom of the flexible catheter or other key components of the system. The above exceptions (i)-(vii) may be considered type I exceptions, whereas exceptions (viii)-(xiii) may be considered type II exceptions. Exception (xiv) may be either of type I or of type II, depending on the intended action of the external command.

In operation 708, the operating context may be determined to assess whether the exception needs to be addressed. The operating context may be relevant because an event may be innocuous in one scenario but may require attention in another scenario. For example, a first operating context may include a setup phase of the system prior to performing a medical procedure. During the setup phase, a malfunction does not put the patient at risk. A second operating context may include an ongoing medical procedure on the patient. During the ongoing medical procedure, the same malfunctions that are considered innocuous during the setup phase may be problematic. The operating context may be detected based on the current configuration of the system. For example, when the swivel connector is docked to the manipulator assembly, the assumption may be that a medical procedure is ongoing or about to be performed. In contrast, when the swivel connector is not docked, the assumption may be that the system is being set up. As another example, attachment and/or extension of the catheter guide to a distal portion of the manipulator assembly may indicate that a medical procedure is ongoing or about to be performed. On the other hand, if the catheter guide is in the retracted position and/or is detached from the distal portion of the manipulator assembly, it may be determined that the system is being set up. The exceptions of the types (iv)-(vii) may be ignored while in the first operating context (e.g., the system is not docked to the patient), whereas they may require attention in the second operating context (e.g., when the system is docked to the patient). Other types of exceptions may require attention regardless of the operating context, for example, when a component is found to be defective.

If the operating context does not require the exception to be addressed (operation 708: no), the method may continue with the execution of operation 702. However, if the operating context necessitates the exception to be addressed (operation 708: yes), the method may proceed with the execution of operation 710.

In operation 710, a distinction may be made between exceptions that may be handled while maintaining catheter articulation, and exceptions that would benefit from relaxing the flexible catheter along the articulation degrees of freedom. Exceptions that may be handled while maintaining the catheter articulation include, for example, exceptions of type (iv), when the trackball and/or scroll wheel is temporarily removed, such as for cleaning. Exceptions that may be handled while maintaining the catheter articulation may also include other types of operational exceptions such as a signal loss or other malfunctions or disruptions that are not directly associated with tracking the motion of the flexible catheter. If an exception that may be handled while maintaining catheter articulation is detected, the method may proceed to a hold state 712 (operations shown in FIG. 7B). If an exception that would benefit from relaxing the flexible catheter along the articulation degrees of freedom is detected, the method may proceed to a control state 718 (operations shown in FIG. 7C).

In the hold state, shown in FIG. 7B, Operations 740, 742, and/or 744 may be performed. When in the hold state, the actuators associated with an articulation degree(s) of freedom and an insertion degree(s) of freedom of the flexible catheter may remain controlled (e.g., servo-controlled), and receive a command to hold the current position of the flexible catheter. The hold state may serve as a landing state in response to certain faults or runtime conditions.

In operation 740, the flexible catheter is controlled to hold the current position, along one or more articulation degrees of freedom. If multiple actuators (e.g., two actuators) are configured to operate in an antagonistic manner, the holding of the current position may be coordinated between the actuators. In one or more embodiments, the control of the one or more articulation degrees of freedom is performed analogous to the control of operation 702, e.g., when operating in a driving mode. However, unlike in operation 702 where the user inputs provided via the input interface may form a command signal causing corresponding movements of the flexible catheter, in operation 740 the command signal is a constant position. Command signals that are received from the input interface (e.g., a trackball or scroll wheel) may be ignored.

In operation 742, the flexible catheter may be controlled to hold a current position along the insertion degree of freedom. Other command signals, such as a command signal to insert or retract the flexible catheter, may be ignored.

Operations 740 and 742 may be simultaneously executed, e.g., after completion of operation 710. For example, the flexible catheter may be controlled along the insertion degree of freedom to hold the current position, while also being controlled to maintain the current catheter articulation.

Commands for flexible catheter movement, for example teleoperation control commands from the input device to articulate the distal portion and/or to insert or retract the flexible catheter, may be ignored. In a scenario that involves a cleaning of the trackball and/or scroll wheel, teleoperation control commands that may be erroneously generated during the removal, cleaning, and reinsertion of the trackball and/or scroll wheel are, thus, ignored. However, the system may still respond to other commands such as clutching commands (e.g., for the setup joints, the insertion axis, etc.).

In operation 744, a notification may be provided to the user, indicating that the system is in the hold state. The notification may clarify to the user that, at this point, teleoperation of the flexible catheter might not be possible. The notification may also indicate the exception that occurred. In the scenario involving the cleaning of the trackball and/or scroll wheel, instructions to remove, clean, and/or reinsert the trackball and/or scroll wheel may be displayed.

Returning to FIG. 7A, in operation 714, the supervisor state machine may continue to monitor the system for events. The supervisor state machine may conclude that an exception has been addressed, for example, when the exception that caused the transition into the control state disappears. For example, in the scenario involving the cleaning of the trackball and/or scroll wheel, a reinsertion of the trackball and/or scroll wheel may have been detected. Additionally or alternatively, another event that addresses the exception may be a fault recovery signal, which may be provided by a user pressing a fault recovery button. If the exception is addressed (operation 714: yes), the system may proceed with operation 716.

Operation 716 may impose a delay, before allowing a return to operation 702.

The delay, in one or more embodiments, may ensure that, in the scenario involving the cleaning of the trackball and/or scroll wheel, the trackball and/or the scroll wheel have been fully reinserted and are in a configuration ready to capture a user's input. Consider, for example, an optical method for detecting presence or absence of the trackball. When the trackball is removed, ambient light may reach a photosensor. The photosensor may thus indicate that the trackball has been removed. Upon reinsertion, the trackball being reinserted may block the ambient light, even before being fully inserted. In this case, immediately allowing the system to return to the operating state may be undesirable, because erroneously generated teleoperation control commands during completion of the reinsertion could cause unwanted movement in one or more degrees of freedom of the flexible catheter. The delay of operation 714 may prevent this undesirable situation. The delay may be sufficiently long to allow full reinsertion of the trackball and/or scroll wheel, into a stable, final position. For example, the delay may be a few hundred milliseconds.

Upon completion of operation 716, the system may return to operation 702.

The return to operation 702 may involve the execution of a fault recovery sequence. Along this sequence, the supervisor state machine may eventually expect a confirmation indicating that the recovery has been successful. If the exception has not been addressed (operation 714: no), the system may remain in the hold state, e.g., by returning to operation 712.

Continuing with the discussion of operation 710, if an exception that would benefit from relaxing the flexible catheter along the articulation degrees of freedom is detected, the system may proceed to a control state 718. In the control state, shown in FIG. 7C, operations 750, 752, and/or 754 may be performed. When in the control state, the system may remain controllable in a “conservative” mode that may be considered safe under a variety of circumstances, as discussed below. In some examples, feedback from the shape sensor might no longer be relied upon in the control state. In the control state, the actuator(s) associated with an articulation degree(s) of freedom of the flexible catheter might not be deactivated, but instead may remain servo-controlled using the system's remaining capabilities to achieve a more graceful and controlled transition, as discussed below. The control state may serve as a landing state in response to certain faults or runtime conditions.

In operation 750, the flexible catheter may be gradually relaxed along one or more articulation degrees of freedom. The relaxation may be performed by gradually reducing the tension in the pull wires. For example, the relaxation may be performed by gradually reducing the current commanded to the actuator associated with the articulation degree of freedom. A ramp-like current reduction may be commanded. If multiple actuators (e.g., two actuators) are configured to operate in an antagonistic manner, the current reduction may be coordinated between the actuators to obtain a relaxation with no or limited articulation movement during the relaxation. In operation 750, the actuator(s) may be under the control of local feedback (e.g., using a position signal obtained from an encoder of the actuator). The local feedback loop may enable a local position control of the actuator, allowing the tension in the pull-wires to be reduced while avoiding significant movement of the articulated portion being controlled by the pull wire. A specified minimum tension may be maintained to prevent the pull-wires from going slack under expected load conditions. In this way, a recovery may later be made without having to wait for a pre-tensioning procedure, which may also induce unwanted motion of the articulated portion or other portions of the flexible catheter. The minimum tension level set by the control algorithms is a value that may depend on the catheter design. For example, if the flexible catheter is equipped with mechanical preloading springs for the pull-wires, the commanded minimum tension may be zero. Other command signals, such as a command signal to articulate the flexible catheter, may be ignored.

In operation 752, the flexible catheter may be controlled to hold a current position along the insertion degree of freedom. Other command signals, such as a command signal to insert or retract the flexible catheter, may be ignored.

Operations 750 and 752 may be simultaneously executed, e.g., after completion of operation 710. For example, the flexible catheter may be controlled along the insertion degree of freedom to hold the current position, while the flexible catheter begins to relax along the articulation degree(s) of freedom.

During the execution of operations 750 and 752, the flexible catheter may gradually become limp along the articulation degrees of freedom, and may be positionally controlled to hold the current position along the insertion degree of freedom. Because the positional locking along the insertion degree of freedom is performed under control rather than by cutting power and engaging a brake, small unwanted motions along the insertion degree of freedom until the flexible catheter comes to rest may be minimized or eliminated. Accordingly, the control state may be particularly beneficial when operating in delicate environments.

Commands for flexible catheter movement, for example teleoperation control commands from the input device to articulate the distal portion and/or to insert or retract the flexible catheter, may be ignored. However, the system may still respond to other commands such as clutching commands (e.g., for the setup joints, the insertion axis, etc.).

In operation 754, a notification may be provided to the user, indicating that the system is in the control state. The notification may clarify to the user that, at this point, teleoperation of the flexible catheter might not be possible. The notification may also indicate the exception that occurred (e.g., a distal sensor data exception, excessive articulation actuator travel exception, input device missing exception, catheter guide exception, etc.).

Returning to FIG. 7A, in operation 720, the supervisor state machine may continue to monitor the system for events. The supervisor state machine may conclude that an exception has been addressed, for example, when the exception that caused the transition into the control state disappears. For example, if the event is a swivel connector undocked exception, proper docking of the swivel connector may resolve the exception. Additionally or alternatively, another event that addresses the exception may be a fault recovery signal, which may be provided by a user pressing a fault recovery button. If the exception is addressed (operation 720: yes), the execution of the method may return to operation 702. The return to operation 702 may involve the execution of a fault recovery sequence. Along this sequence, the supervisor state machine may eventually expect a confirmation indicating that the recovery has been successful. If the exception has not been addressed (operation 720: no), the system may remain in the control state, e.g., by returning to operation 718.

Operation 722 may be the landing state in response to certain exceptions, as previously discussed with reference to operations 704 and 706. For example, operation 722 may be performed if a type II exception is detected. In operation 722, the system may enter a fault reaction logic state. In the fault reaction logic state, the flexible catheter may be locked along the insertion degree of freedom. Safety brakes may be applied. Power may be removed from the insertion actuator(s), and brake(s) of the actuator(s) may be engaged. In addition, the motor leads of the actuator(s) may be shorted to produce additional electrical damping. Power may also be removed from the actuator(s) associated with the articulation degree of freedom. Accordingly, the actuator(s) might no longer be servo-controlled. The non-servo-controlled actuators might no longer respond to input commands, and the flexible catheter may be allowed to relax (e.g., go limp) immediately (e.g., rather than gradually).

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the claims.

What is claimed is:

1. A medical system, comprising:
a flexible catheter, comprising a flexible body having a proximal portion and a distal portion, wherein the distal portion is articulable along an articulation degree of freedom of the flexible catheter;
an instrument carriage supporting the flexible catheter at the proximal portion of the flexible body;
a first actuator disposed on the instrument carriage and configured to drive the distal portion of the flexible catheter along the articulation degree of freedom; and
a controller coupled to the first actuator, the controller configured to:
detect an exception of the medical system,
based on detection of the exception, switch from an operating state to a control state, and
control the first actuator, in the control state, to relax the flexible catheter along the articulation degree of freedom.

2. The medical system of claim 1, further comprising a second actuator configured to drive the instrument carriage to cause movement of the flexible catheter along an insertion degree of freedom.

3. The medical system of claim 2, wherein the controller is further configured to:
when in the control state, hold a current position of the flexible catheter along the insertion degree of freedom.

4. The medical system of claim 2, further comprising an input device configured to receive commands, wherein the controller is further configured to, when in the control state:
ignore one or more commands received by the input device from a user, wherein the one or more commands is for at least one of the first actuator or the second actuator.

5. The medical system of claim 2, wherein the controller is further configured to:
detect a second exception, and
based on detection of the second exception, prevent motion of the flexible catheter along the insertion degree of freedom.

6. The medical system of claim 5, wherein preventing motion of the flexible catheter along the insertion degree of freedom comprises:
engaging a brake of the second actuator.

7. The medical system of claim 1, wherein the controller is further configured to:
issue a notification that the medical system is in the control state.

8. The medical system of claim 1, wherein the exception comprises a system error associated with the medical system.

9. The medical system of claim 8,
wherein the flexible catheter further comprises a shape sensor for determining a shape of at least a portion of the flexible body, and
wherein the system error comprises a malfunction of the shape sensor.

10. The medical system of claim 1, wherein the exception comprises a user-induced exception.

11. The medical system of claim 1, wherein the exception is triggered by at least one of:
a removal of an anti-buckling guide associated with the flexible catheter,
an unlocking of at least one setup joint, or
an undocking of a swivel connector.

12. The medical system of claim 1,
wherein detection of the exception occurs under a first operating context, and
wherein detection of the exception under a second operating context causes the medical system to continue operating in the operating state.

13. The medical system of claim 12,
wherein the first operating context comprises an ongoing medical procedure, and
wherein the second operating context comprises a setup phase, prior to the ongoing medical procedure.

14. The medical system of claim 1, wherein controlling the first actuator to relax the flexible catheter comprises controlling the first actuator to gradually relax the flexible catheter along the articulation degree of freedom or immediately relax the flexible catheter along the articulation degree of freedom.

15. The medical system of claim 1, wherein controlling the first actuator to relax the flexible catheter along the articulation degree of freedom comprises commanding a reduction of current for the first actuator.

16. The medical system of claim 15, wherein the reduction of current comprises a ramped reduction of current.

17. The medical system of claim 1,
wherein the distal portion of the flexible body is coupled to the first actuator by a control element, and
wherein controlling the first actuator to relax the flexible catheter along the articulation degree of freedom comprises maintaining the control element at a minimum tension.

18. The medical system of claim 1, wherein the controller is further configured to:
detect a second exception of the medical system,
based on detection of the second exception, switch from the operating state to a hold state, and
control the first actuator, in the hold state, to maintain a current articulation of the flexible catheter along the articulation degree of freedom.

19. The medical system of claim 18, wherein the controller is further configured to:
based on a determination that the second exception is no longer detected and after a delay, switch from the hold state to the operating state.

20. The medical system of claim 18, wherein the second exception is triggered by a removed input device configured to receive commands from a user.
